(19) United States
(12) Patent Application Publication
(10) Pub. No.: US 2022/0005557 A1
(43) Pub. Date: Jan. 6, 2022

(54) SYSTEM AND METHOD FOR RECOMMENDING PROTOCOL USED FOR TISSUE PROCESSING

(71) Applicant: LEICA BIOSYSTEMS NUSSLOCH GMBH, Nussloch (DE)

(72) Inventors: Li YIN, Shanghai (CN); Qi CHENG, Shanghai (CN)

(21) Appl. No.: 17/279,236

(22) PCT Filed: Nov. 7, 2019
(86) PCT No.: PCT/EP2019/080499

§ 371 Date: Mar. 24, 2021

(30) Foreign Application Priority Data
Nov. 9, 2018 (CN) 201811330703.5

(51) Int. Cl. G16H 10/40; G16H 50/20; G16H 40/20; G16H 70/20; G06K 7/14; G06K 7/10; G06F 16/245; G01N 1/31

(52) U.S. Cl. CPC G16H 10/40; G16H 50/20; G16H 40/20; G16H 70/20; G06K 7/1413; G01N 2001/315; G06K 7/10297; G06K 7/10861; G06F 16/245; G01N 1/31; G06K 2007/10504; G06K 7/1417

(57) ABSTRACT

A system and a method for recommending a protocol used for tissue processing are provided. The system includes: a basket, attached with an identification code, wherein the identification code is configured to identify a type of tissue contained in a cassette carried by the basket; and a tissue processor, configured to read the identification code to obtain the type of tissue, and to recommend a protocol according to the obtained type of tissue. With the system, it is possible to automatically recommend the protocol suitable for tissue processing, avoiding that a user selects a wrong protocol, and improving a quality of the tissue processing.

### FIELD

The present invention relates to a technology field of tissue processor, and more particularly to a method and a system for recommending a protocol used for tissue processing.

### BACKGROUND

A tissue processor is a device that automatically immerses tissue samples in a series of solvents in a reactor to process (e.g., immobilize, dehydrate, transparentize, wax, etc.) the tissue samples. An appropriate protocol needs to be used in the process, depending on the type of the tissue.

### SUMMARY

The present invention aims to solve at least one of problems in the related art to at least some extent.

Accordingly, a first objective of the present invention is to provide a system for recommending a protocol used for tissue processing, which may automatically recommend the protocol suitable for use in the tissue processing, thereby avoiding that a user selects a wrong protocol, and improving a quality of the tissue processing.

A second objective of the present invention is to provide a method for recommending a protocol used for tissue processing.

In order to achieve above objectives, embodiments of the first aspect of the present invention provide a system for recommending a protocol used for tissue processing. The system includes: a basket, attached with an identification code, wherein the identification code is configured to identify a type of tissue contained in a cassette carried by the basket; and a tissue processor, configured to read the identification code to obtain the type of tissue, and to recommend a protocol according to the obtained type of tissue.

With the system according to embodiments of the present invention, by attaching to the basket, the identification code identifying the type of tissue contained in a cassette carried by the basket, and by reading the identification code, it is possible to automatically recommend the protocol suitable for use in the tissue processing, thereby avoiding that the user selects a wrong protocol, and improving a quality of the tissue processing.

In an embodiment of the present invention, the tissue processor includes: a reader, configured to read the identification code to obtain the type of tissue; a recommendation unit, configured to recommend the protocol according to the obtained type of tissue and a pre-stored relation table between types of tissues and protocols; and a display, configured to display recommended protocol.

In an embodiment of the present invention, the tissue processor further incudes: an input unit, configured to, when a plurality of protocols is recommended, receive an input from a user to select one of the plurality of protocols for tissue processing.

In an embodiment of the present invention, the system for recommending the protocol used for tissue processing further includes a laboratory information management system. The laboratory information management system is configured to store a correspondence relationship between baskets and types of tissues in the cassettes. The tissue processor further includes an interface module. The interface module is configured to connect the tissue processor with the laboratory information management system, such that the tissue processor obtains the type of tissue contained in the cassette carried by the basket by querying the laboratory information management system.

In an embodiment of the present invention, the basket is provided with a particular color, the color representing the type of the tissue. Therefore, the user may know the type of the tissue corresponding to the basket based on the particular color, and put the cassette containing the tissue of this type into the basket.

In an embodiment of the present invention, the identification code attached to the basket comprises at least one of s a bar code, a QR code and an RFID tag.

In order to achieve above objectives, embodiments of the present invention provide a method for recommending a protocol used for tissue processing. The method includes: reading an identification code attached on a basket carrying a cassette, to obtain a type of tissue contained in the cassette; and recommending at least one protocol according to the type of tissue.

With the method according to embodiments of the present invention, by attaching to the basket, the identification code identifying the type of tissue contained in a cassette carried by the basket, and by reading the identification code, it is possible to automatically recommend the protocol suitable for use in the tissue processing, thereby avoiding that the user selects a wrong protocol, and improving a quality of the tissue processing.

In an embodiment of the present invention, recommending the protocol according to the type of tissue includes: recommending the protocol according to the type of tissue and a pre-stored relation table between types of tissues and protocols; and displaying recommended protocol.

In an embodiment of the present invention, the method further includes: when a plurality of protocols is recommended, receiving an input from a user to select one of the plurality of protocols for tissue processing.

In an embodiment of the present invention, obtaining the type of tissue contained in the cassette includes: querying a laboratory information management system to obtain the type of tissue contained in the cassette, in which the laboratory information management system stores a correspondence relationship between baskets and types of tissues in the cassettes.

In an embodiment of the present invention, the method further includes, especially before reading the identification code attached on the basket carrying the cassette: determining the type of tissue corresponding to the basket according to a color of the basket; and loading the cassette containing tissue having the type corresponding to the basket into the basket. In such an embodiment, the basket is provided with a particular color, the user may know the type of tissue corresponding to the basket according to the color of the basket, and thus may load the cassette containing tissue having the type corresponding to the basket into the basket.

In an embodiment of the present invention, the identification code comprises at least one of a bar code, a QR code and an RFID tag.

### BRIEF DESCRIPTION OF THE DRAWINGS

FIG. 1 is a block diagram of a system for recommending a protocol used for tissue processing according to an embodiment of the present invention;

FIG. 2 is a block diagram of a tissue processor according to an embodiment of the present invention;

FIG. 3 is a block diagram of a tissue processor according to an embodiment of the present invention;

FIG. 4 is a block diagram of a system for recommending a protocol used for tissue processing according to an embodiment of the present invention; and

FIG. 5 is a flow chart of a method for recommending a protocol used for tissue processing according to an embodiment of the present invention.

### DETAILED DESCRIPTION

Reference will be made in detail to embodiments of the present invention. Embodiments of the present invention will be shown in drawings, in which the same or similar elements and the elements having same or similar functions are denoted by like reference numerals throughout the descriptions. The embodiments described herein according to drawings are explanatory and illustrative, not construed to limit the present invention.

In the related art, when a tissue processor is used to process tissue samples, a user is required to manually select the protocol to be used for tissue processing based on paper record. The user needs to manually record the type of tissue contained in the cassette carried in the basket during grossing, and needs to identify the basket and find the type of tissue from the paper record during the tissue processing.

However, paper record is very inefficient and may not prevent the user from selecting the wrong protocol. Embodiments of the present invention therefore provide a system and method for automatically recommending a protocol suitable for use in tissue processing.

FIG. 1 is a block diagram of a system for recommending a protocol used for tissue processing according to an embodiment of the present invention. As illustrated in FIG. 1, the system includes a basket 10 and a tissue processor.

The basket 10 is attached with an identification code 11. The identification code 11 is configured to identify a type of tissue contained in a cassette carried by the basket 10. The tissue processor 20 is configured to read the identification code 11 to obtain the type of tissue, and to recommend a protocol according to the obtained type of tissue. For example, for small tissue, a standard 2-hour procedure may be recommended, and for big tissue, an approved overnight procedure may be recommended.

With the system according to embodiments of the present invention, by attaching to the basket 10, the identification code 11 identifying the type of tissue contained in the cassette carried by the basket 10, and by reading the identification code 11, it is possible to automatically recommend the protocol suitable for use in tissue processing, thereby avoiding that the user selects a wrong protocol, and improving a quality of the tissue processing.

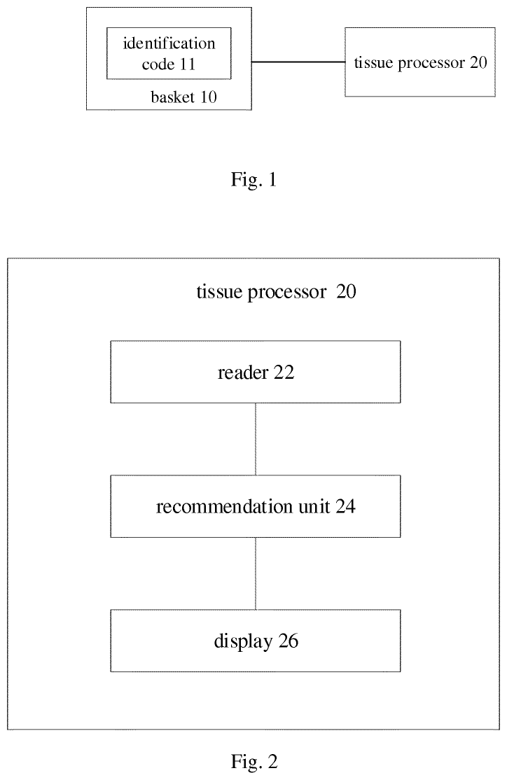

In detail, as illustrated in FIG. 2, the tissue processor may include a reader 22, a recommendation unit 24 and a display 26. The reader 22 is configured to scan the identification code 11 to obtain the type of tissue. The recommendation unit 24 is configured to recommend the protocol according to the obtained type of tissue and a pre-stored relation table between types of tissues and protocols. The display 26 is configured to display recommended protocol.

In an embodiment of the present invention, the identification code 11 may be realized in various forms. For example, the identification code 11 may be realized as a bar code, a QR code or a RFID tag. Accordingly, the reader 22 may be realized as a scanner or a RFID reader. When the identification code 11 is a bar code or a QR code, the scanner may scan the bar code or the QR code to obtain the type of tissue. When the identification code 11 is the RFID tag, the RFID reader may read the RFID tag to obtain the type of tissue.

In an embodiment of the present invention, a correspondence relationship between types of tissues and protocols used for processing tissues of these types may be established in advance, to generate a relation table, and the relation table may be stored in the tissue processor. When the identification code of the basket is read to obtain the type of tissue, a recommended protocol may be obtained by querying the relation table. It should be understood that one or more protocols may be recommended for one type of tissue. When more than one protocols are recommended, the user may select one from the recommended protocols for tissue processing.

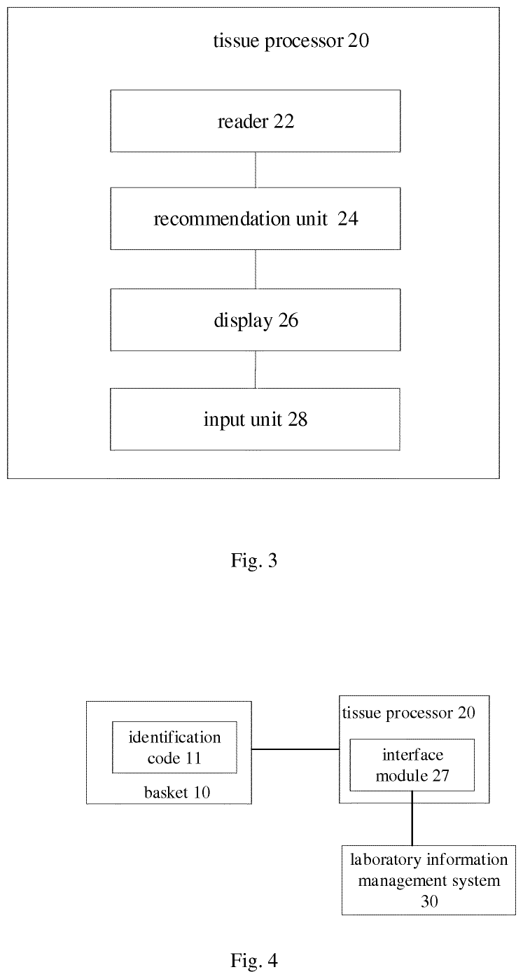

As illustrated in FIG. 3, the tissue processor 20 may further include an input unit 28. The input unit 28 is configured to, when a plurality of protocols is recommended, receive an input from the user to select one of the plurality of protocols for tissue processing. For example, the input unit 28 may include, but is not limited to, a touch screen and a mouse.

As illustrated in FIG. 4, the system for recommending the protocol used for tissue processing may further include a laboratory information management system 30. The laboratory information management system 30 is configured to store a correspondence relationship between baskets and types of tissues in the cassettes. The tissue processor 20 may include an interface module 27. The interface module 27 is configured to connect the tissue processor 20 with the laboratory information management system 30, such that the tissue processor 20 may obtain the type of tissue contained in the cassette carried by the basket 10 by querying the laboratory information management system 30.

In an embodiment of the present invention, when the user grossing tissue samples, the user may determine the type of tissue corresponding to the basket 10 based on a color of the basket 10, and load the tissue sample of correct type to the basket.

In an embodiment of the present invention, the basket 10 is provided with a particular color. The color represents the type of tissue. The user may easily and quickly know the type of tissue corresponding to the basket 10 by visual observation, and thus the user may load the cassette containing tissue of the type into the basket. Further, the tissue processor 20 may obtain the type of tissue corresponding to the basket 10 correctly and recommend the appropriate protocol.

In conclusion, with the system according to embodiments of the present invention, the user may load the cassette containing a certain type of tissue into the corresponding basket 10, and the tissue processor 20 may obtain the type of tissue contained in the cassette carried by the basket 10 by reading the identification code 11 attached to the basket 10, and thus may automatically recommend the protocol suitable for use in the tissue processing, thereby avoiding that the user selects the wrong protocol, and improving the quality of the tissue processing.

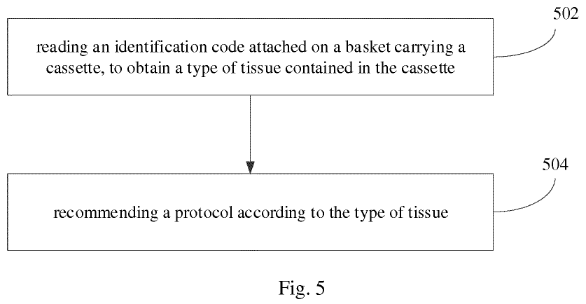

Embodiments of the present invention further provide a method for recommending a protocol used for tissue processing. As illustrated in FIG. 5, the method may include followings.

At block 502, an identification code attached on a basket carrying a cassette is read, to obtain a type of tissue contained in the cassette.

At block 504, a protocol is recommended according to the obtained type of tissue.

In an embodiment of the present invention, the identification code may be realized as a bar code, a QR code or a RFID tag. Therefore, the identified code may be read by scanning the bar code or QR code or by reading the RFID tag, and further the type of tissue may be obtained.

In an embodiment of the present invention, a correspondence relationship between types of tissues and protocols used for processing tissues of these types may be established in advance, to generate a relation table. When the identification code of the basket is read to obtain the type of tissue, a recommended protocol may be obtained by querying the relation table. It should be understood that one or more protocols may be recommended for one type of tissue. When more than one protocol are recommended, the user may select one from the recommended protocols for tissue processing.

In an embodiment of the present invention, the type of tissue contained in the cassette carried by the basket may be obtained by querying a laboratory information management system. In detail, a correspondence relationship between baskets and types of tissues in the cassettes is stored in the laboratory information management system. Thus, the number of the basket may be obtained by reading the identification code, and then the type of tissue contained in the cassette may be obtained according to the number of the basket.

In an embodiment of the present invention, when the user grossing tissue samples, the user may determine the type of tissue corresponding to the basket based on a color of the basket, and load the tissue sample of correct type to the basket.

In an embodiment of the present invention, before reading the identification code attached on the basket carrying the cassette, the method further includes: determining the type of tissue corresponding to the basket according to the color of the basket; and loading the cassette containing tissue having the type corresponding to the basket into the basket. In such an embodiment, the basket is provided with a particular color, the color representing the type of tissue. The user may easily and quickly know the type of tissue corresponding to the basket by visual observation, and thus the user may load the cassette containing tissue of the type into the basket. Further, the type of tissue corresponding to the basket may be obtained correctly and the appropriate protocol may be recommended.

With the method according to embodiments of the present invention, the user may load the cassette containing a certain type of tissue into the corresponding basket, and the type of tissue contained in the cassette carried by the basket may be obtained by reading the identification code attached to the basket, and thus the protocol suitable for use in the tissue processing may be automatically recommended, thereby avoiding that the user selects the wrong protocol, and improving the quality of the tissue processing.

It is to be understood that, in the description, terms such as “first” and “second” are used herein for purposes of description and are not intended to indicate or imply relative importance or significance. Thus, the feature defined with “first” and “second” may comprise one or more this feature. In the description of the present invention, “a plurality of” means two or more than two, unless specified otherwise.

In the present utility model, unless specified or limited otherwise, the terms “mounted,” “connected,” “coupled,” “fixed” and the like are used broadly, and may be, for example, fixed connections, detachable connections, or integral connections; may also be mechanical or electrical connections; may also be direct connections or indirect connections via intervening structures; may also be inner communications of two elements, which can be understood by those skilled in the art according to specific situations.

Reference throughout this specification to “an embodiment,” “some embodiments,” “one embodiment”, “another example,” “an example,” “a specific example,” or “some examples,” means that a particular feature, structure, material, or characteristic described in connection with the embodiment or example is included in at least one embodiment or example of the present utility model. Thus, the appearances of the phrases such as “in some embodiments,” “in one embodiment”, “in an embodiment”, “in another example,” “in an example,” “in a specific example,” or “in some examples,” in various places throughout this specification are not necessarily referring to the same embodiment or example of the present utility model. Furthermore, the particular features, structures, materials, or characteristics may be combined in any suitable manner in one or more embodiments or examples.

Although explanatory embodiments have been shown and described, it would be appreciated by those skilled in the art that the above embodiments cannot be construed to limit the present utility model, and changes, alternatives, and modifications can be made in the embodiments without departing from spirit, principles and scope of the present invention.

1. A system for recommending a protocol used for tissue processing, comprising:
a basket (10), attached with an identification code (11), wherein the identification code is configured to identify a type of tissue contained in a cassette carried by the basket; and
a tissue processor (20), configured to read the identification code to obtain the type of tissue, and to recommend a protocol according to the obtained type of tissue, wherein the tissue processor (20) comprises:
a reader, configured to read the identification code to obtain the type of tissue;
a recommendation unit, configured to recommend the protocol according to the obtained type of tissue and a pre-stored relation table between types of tissues and protocols;
a display, configured to display recommended protocol; and
an input unit, configured to, when a plurality of protocols is recommended, receive an input from a user to select one of the plurality of protocols for tissue processing.

2. (canceled)

3. (canceled)

4. The system according to claim 1, further comprising:
a laboratory information management system (30), configured to store a correspondence relationship between baskets and types of tissues in the cassettes,
wherein the tissue processor (20) comprises:
an interface module (27), configured to connect the tissue processor with the laboratory information management system (30), such that the tissue processor obtains (original) the type of tissue contained in the cassette carried by the basket (20) by querying the laboratory information management system (30).

5. The system according to claim 1, wherein the basket (10) is provided with a particular color, the color representing the type of the tissue.

6. The system according to claim 1, wherein the identification code (11) comprises at least one of a bar code, a QR code and an RFID tag.

7. A method for recommending a protocol used for tissue processing, comprising:
reading an identification code (11) attached on a basket (10) carrying a cassette, to obtain a type of tissue contained in the cassette; and
recommending a protocol according to the type of tissue, wherein recommending the protocol according to the type of tissue comprises recommending the protocol according to the type of tissue and a pre-stored relation table between types of tissues and protocols, and displaying recommended protocol; and
when a plurality of protocols is recommended, receiving an input from a user to select one of the plurality of protocols for tissue processing.

8. (canceled)

9. (canceled)

10. The method according to claim 7, wherein obtaining the type of tissue contained in the cassette comprises:
querying a laboratory information management system (30) to obtain the type of tissue contained in the cassette, in which the laboratory information management system stores a correspondence relationship between baskets and types of tissues in the cassettes.

11. The method according to claim 7, wherein the identification code comprises at least one of a bar code, a QR code and an RFID tag.

12. The method according to claim 7, further comprising:
determining the type of tissue corresponding to the basket according to a color of the basket; and
loading the cassette containing tissue having the type corresponding to the basket into the basket.
